(19) United States
(12) Patent Application Publication
(10) Pub. No.: US 2020/0147307 A1
(43) Pub. Date: May 14, 2020

(54) SYSTEMS AND METHOD FOR DETECTION AND TREATMENT OF OPIOID OVERDOSE

(71) Applicants: Steven Yap, Long Beach, NY (US); David Nogueras Manquillo, London (GB); Vasiliki Kiparoglou, Oxford (GB); Mauricio Lopez Santaella, Meinerzhagen (DE); William Yap, Long Beach, NY (US)

(72) Inventors: Steven Yap, Long Beach, NY (US); David Nogueras Manquillo, London (GB); Vasiliki Kiparoglou, Oxford (GB); Mauricio Lopez Santaella, Meinerzhagen (DE); William Yap, Long Beach, NY (US)

(73) Assignee: Dr. Steven Yap, Long Beach, NY (US)

(21) Appl. No.: 16/188,258

(22) Filed: Nov. 12, 2018

(51) Int. Cl. A61M 5/20; A61M 5/172; A61B 5/16

(52) U.S. Cl. CPC A61M 5/2046; A61M 5/2033; C07D 489/12; A61B 5/16; A61M 2005/206; A61M 5/1723

(57) ABSTRACT

A device and method utilizing user operated actuators such as momentary switches, intermediate switches, accelerometers or video cameras to detect impaired consciousness as an early symptom of opioid overdose to then trigger administration of preloaded naloxone to rescue the user from an overdose.

### CROSS REFERENCES TO RELATED APPLICATIONS

This application claims the benefit of Applicants' prior provisional application, number 62/584,867, filed on Nov. 12, 2017.

### TECHNICAL FIELD

The present invention relates generally to systems and methods for opioid overdose detection and rescue.

### BACKGROUND OF THE INVENTION

### Background

Opioid overdose is an acute condition resulting from excessive intake of opioids such as morphine, oxycodone, fentanyl or heroin and was directly implicated in the death of 52,000 Americans in 2016. The sequence of events in death from opioid overdose typically progresses from euphoria to loss of consciousness, respiratory arrest, cardiac arrest and ultimately death.

Naloxone is a potent opioid antagonist that reverses the effects of opioids by restoring consciousness and breathing. The value of Naloxone in reducing death from opioid overdose is widely acknowledged: naloxone preparations in vial, syringe, nasal spray and autoinjector (pre-loaded mechanical injector) formats have been increasingly distributed as a stop-gap measure.

However, in an opioid overdose, the victim is unconscious and current naloxone formulations require an adjacent bystander to correctly diagnose the overdose and administer naloxone to the victim. There are no commercially viable naloxone self-rescue devices available.

This is alarming as most opioids are abused in the privacy of the home where a bystander may not be present. The bystander requirement may be limiting naloxone's life saving potential.

### Current Teaching

In patients at high risk for opioid overdose in the medical setting, current and accepted medical practice is to continuously monitor vital signs to warn of emergent deterioration of a patient's clinical status. In the setting of an acute overdose, naloxone is then injected by a nurse or physician. Medical vital signs are used as surrogates for respiration (pulse oximetry and respiratory rate as measured by end tidal carbon dioxide or transthoracic impedance) and circulation (blood pressure and EKG).

Recent attempts to develop opioid overdose self-rescue devices and methods (Donnellan, Insler, Olson and Nielsen) all teach towards the mimicry and automation of current and accepted medical practice in which sensors are used to continuously monitor vital signs for aberrancy, which then triggers injection of preloaded naloxone.

However, failure to rescue in the non-medical drug use setting may result from dependence on delicate vital sign sensors normally intended for use by expert medical practitioners in the healthcare setting.

Pulse oximetry is a medical device that indirectly measures respiratory depression by detection of blood oxygenation levels. Hypoxia is typically defined by a reading below 92% beyond which life-threatening anoxia rapidly ensues within three minutes. However, it should be noted that nasal and intramuscular naloxone requires up to ten minutes to provide effect. Hence, a bystander may still be required to provide rescue breaths. Pulse oximetry measurements requires translucency of the body area may be affected by nail polish, vascular disease cold temperature and movement.

End-Tidal Carbon Dioxide (ETCO2, capnography, capnometry) is an extremely sensitive method for detecting respiratory depression in the clinical setting. There are two methods for ETCO2-mainstream and sidestream. Mainstream capnography requires a tight-fitting mask while sidestream capnography utilizes a disposable cannula that is prone to clogging. Successful measurement and interpretation generally requires medical personnel with specific training. Furthermore, portable capnography is high cost with a three-hour battery life.

Other methods such as heart rate, blood pressure and electrocardiography indicate impending physiological collapse and are very likely to require a bystander to administer rescue breaths and chest compressions. Low blood pressure and low cardiac output delay circulation of injected naloxone to the brain.

### BRIEF SUMMARY OF THE INVENTION

The present invention is directed to a system and method for detecting impaired consciousness as an early sign of opioid overdose and trigger the administration of preloaded naloxone medicament or alert a third party.

Consciousness or wakefulness of the user is monitored by requiring the user to engage or activate actuator(s) specifically designed to require conscious effort. Involuntary disengagement or release of actuator(s) suggests impaired consciousness and results in an alarm. Should the user fail to respond to this alarm by re-engaging the actuator(s), preloaded naloxone is injected automatically to rescue the unconscious user.

Impaired consciousness can also be detected by failure of user to respond to a series of prompts at timed intervals. The actuator may be a motion detection sensor such as an accelerometer or video camera or may utilize facial recognition technology.

Monitoring for impaired consciousness as a surrogate for opioid overdose is distinct from monitoring of vital signs, which aims to detect respiratory and cardiac depression or arrest. Monitoring for impaired consciousness is strategic as it occurs earlier in the cascade of symptoms associated with opioid overdose, thus providing precious additional time for rescue treatment to take effect. Furthermore, this method alleviates the cost, regulatory requirements and technical complexity of ensuring accurate measurement of vital signs and setting appropriate parameters.

### BRIEF DESCRIPTION OF THE DRAWINGS

FIG. 1 shows the embodiment of the invention to detect an opioid overdose and trigger injection of naloxone. The device is worn on the arm or leg, preferably the upper arm or thigh area. The device is held in place using an adjustable strap such that the device is not easily removed inadvertently and is secured in a manner such to allow function of the injector mechanism.

The wearable device contains a battery 104 to power the processor 105 and alarm 106. The alarm may consist of a speaker to emit a loud or unpleasant sound, LED to emit light or a flash of light, vibrator to provide a vibration signal or a combination of these. The device contains buttons to allow the user to turn the device on/off 101 and bypass the alarm to immediately administer naloxone 102. The actuator(s) may be contained in the main device housing 103 and/or contained in a handheld console 110 that may be connected to the wearable device 108 using an electronic cable or via wireless technology. The buttons are designed in such a manner to prevent inadvertently turning off the device or accidentally administer naloxone using the bypass button. The device may contain an automated injector mechanism 107, needle 109 and medicament 111. A variety of automated injector mechanisms can be used for this device including electric powered, gas powered or spring loaded mechanisms, known to those of ordinary skill in the art of automated injectors.

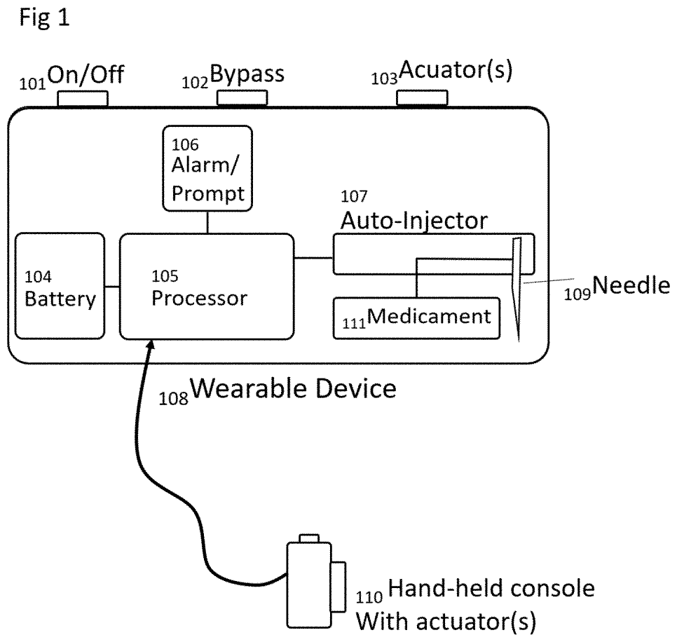

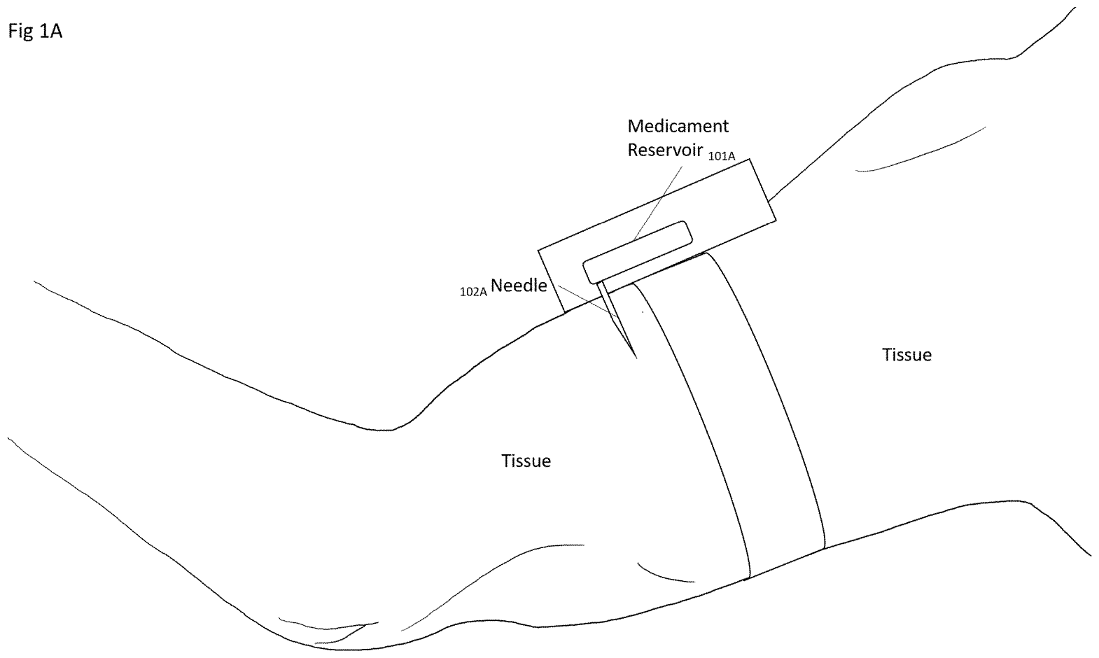

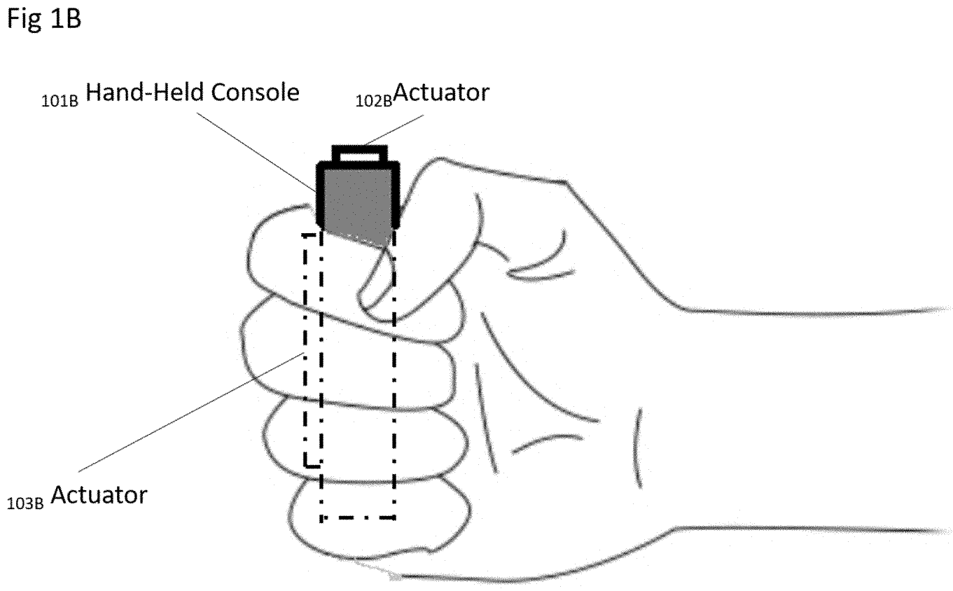

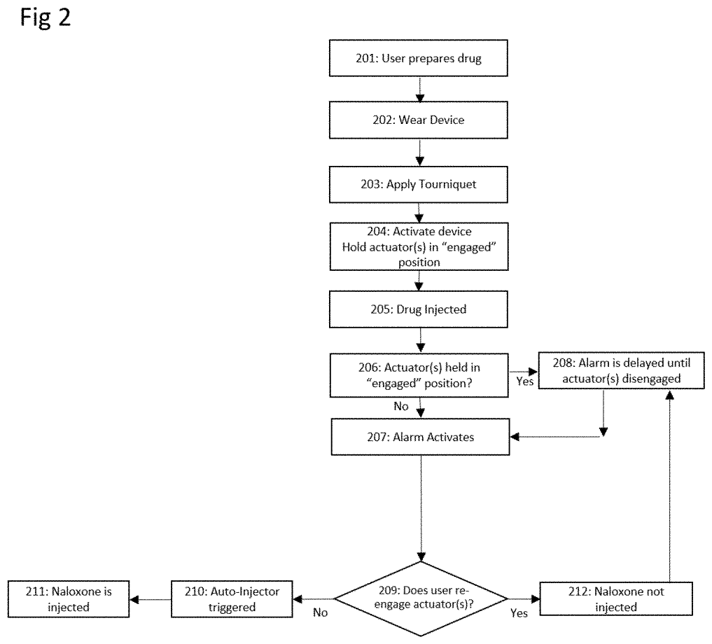

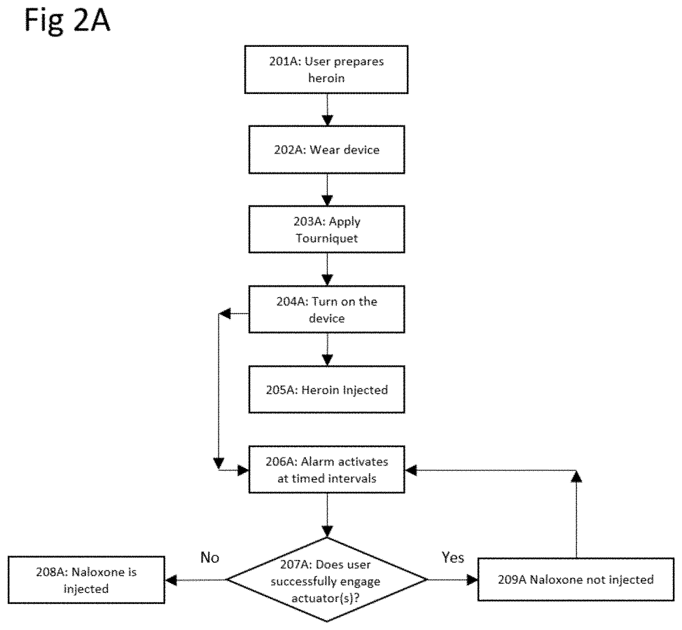

As noted in FIG. 1A, activation of the automated injector mechanism after detection of opioid overdose drives the needle into tissue and injects medicament into the user. The medicament reservoir 101A contains at least one dose of Naloxone to reverse opioid overdose.

As shown in FIG. 1B, the device may require the user to press down on two actuators 102B and 103B on the handheld console 101B to improve reliability.

FIG. 2 is an exemplary flowchart showing the method for detecting impaired consciousness and triggering of naloxone injection in the setting of drug abuse. The user is advised to prepare heroine beforehand 201. The device is worn 202 and tourniquet applied 203. Alternatively, the tourniquet may be integrated into the device. The device is activated by holding actuator(s) in the “engaged” position 204 preferably before injection of drug 205. The user continues to hold the actuator(s) in the “engaged” position 206. A user with impaired unconsciousness due to the effects of drug will be unable to continue engaging actuator(s) 208. Release of the actuator(s) 208 activates alarm 207, requiring the user to re-engage actuator(s) 209. Failure to re-engage actuator(s) triggers automated injector mechanism 210 and injection of Naloxone 211. In contrast, if the user successfully re-engages the actuator(s) in response to the alarm prompt 207, Naloxone is not injected 212 and subsequent alarm is delayed until actuator(s) are again released 208.

FIG. 2a is an exemplary flowchart showing an alternate method for detecting and treating an opioid overdose which utilizes a series of alarm prompts at equally or variably spaced time intervals 206A. The device is worn 202A and tourniquet is applied 203A. The device is activated 204A preferably prior to injection of heroin 205A. Alarm intervals may be determined by user input of data into interface including drug of abuse, route of abuse, quantity abused, estimated potency and demographic data such as gender, age, weight and height. User responsiveness to alarm may also be utilized to adapt the pattern of alarm sequence presented to the user. Delayed response(s) to alarm prompt may be indicative of onset of lethargy or obtundation and may result in decreasing time interval(s) between alarm(s). Alternatively, prompt response(s) may indicate that the pharmacologic effect of heroin is wearing off and may result in lengthening of subsequent alarm interval(s). Should the user fail to intermittently engage actuator(s) in response to an alarm prompt 207A within a time period, the autoinjector mechanism is activated and naloxone is administered 208A. If the user successfully provides negative feedback, naloxone is not injected and after a specific amount of time, the next alarm in the series is presented to the user 206A.

### DETAILED DESCRIPTION OF THE INVENTION

The present invention is directed to a system and method for using actuator(s) to detect impaired consciousness as an early sign of opioid overdose and to trigger the injection of preloaded naloxone or alert a third party. In the primary embodiment, the invention is designed such that a user with impaired consciousness is no longer able to operate the actuators to provide the required feedback to the device.

The device includes a processor, a user interface, a battery, an actuator(s), an automated injection mechanism and at least one dose of preloaded medicament. The device is not limited to naloxone for reversing opioid toxicity and may be utilized for other toxicities such as organophosphate poisoning in which medicament such as atropine is administered. The device may be worn on an arm or leg and may be handheld or implanted. This system and method is intended to be used from the time of initial drug intake until the pharmacologic effect of drug has decreased such that risk of overdose has passed.

In its primary embodiment, the actuator takes the form of a momentary switch that requires continuous effort on the user to hold in the engaged or depressed position. This switch is preferably in a handheld housing. Continuous engagement or depression of the momentary switch signifies consciousness while disengagement or release of the momentary switch suggests possible impaired consciousness, resulting in an alarm alerting the user to re-engage the switch within a time period to prevent triggering the administration of naloxone.

In another embodiment, the device contains at least two actuators in the form of momentary switches which the user must continuously engage or depress. These switches are also preferably in a handheld housing. This feature reduces false negative type errors in the detection of impaired consciousness.

In yet another embodiment, the device contains an actuator(s) in the form of an intermediate switch(es) which the user must continuously maintain in the intermediate range. These switches are also preferably in a handheld housing. Releasing the switch into the open position or engagement of the switch into the closed position suggests impaired consciousness, resulting in an alarm which requires the user to re-engage the switch into the intermediate position to prevent naloxone injection.

In yet another embodiment, the device utilizes actuator(s) in the form of motion detection sensors including accelerometers or video cameras. These sensors are preferably in a medicament housing or may even employ the user's smartphone sensors. Failure for the actuator(s) to sense movement within a predetermined threshold that monitors the quantity of movement over time results in an alarm which requires user movement to prevent naloxone injection.

In yet another embodiment, the device may challenge the user to confirm his or her consciousness by alerting the user through visual, auditory, tactile or haptic prompts to voluntarily disengage and then re-engage the actuator(s).

In yet another embodiment, the device does not require continuous engagement of the actuator but instead presents the user with a series of prompts at timed intervals, to which failure of the user to provide feedback to the device by intermittently engaging the actuator(s) within a time period may result in an alarm and subsequent injection of naloxone. Time interval between prompts may be constant or variable and may be determined in part by delay of user response to previous prompt, typical pharmacokinetic of drugs of abuse or user input into user interface including drug of abuse, route of abuse, quantity abused, estimated potency and demographic data such as gender, age, weight and height.

The user interface includes button(s) that allow the user to turn the device on and input data, as well as actuator(s) for the user to provide feedback. In some embodiments, the actuators are contained within a handheld console connected by wire or wireless technology to the main housing. In other embodiments, the actuators are contained within the medicament housing.

The device also includes a processor which collects user input from the user interface, stores data into memory, activate alarm and prompts and trigger activation of injector mechanism. The alarm and prompts may be in the form of audio, visual, tactile/haptic, or a combination of the preceding stimuli.

The device may also include an injector mechanism and at least one dose of naloxone medicament. Multiple injector mechanisms and medicament reservoirs have already been described and are available on the market. Typically, these devices use pump propulsion or electrical motor mechanism to drive the needle into tissue prior to injection of medicament. In the described device, medicament may be injected into subcutaneous, muscle or bone tissue or may alternatively be sprayed into the nose. Medicament administered include but are not limited to naloxone, flumazenil or atropine.

The device may also include wireless connectivity with a smartphone to contact a third party should impaired consciousness be detected.

1. A method for detecting impaired consciousness to determine the need for automatic administration of medicament to a user suspected of overdosing on an opiate, the method comprising:
a. monitoring of the user's consciousness by the consistent activation of an actuator(s) connected to a device,
b. said device comprises a processor, an interface adapted to interface to said user, and an actuator(s) contained in the device or in a separate housing,
c. determining a conscious user state by the device by detecting a consistent activation of said actuator(s),
d. determining a possible user state of impaired consciousness by the device by detecting a deactivation or inconsistent activation of said actuator(s),
e. triggering an alarm on said device if said possible unconscious state is determined,
f. the alarm requiring the user to re-activate said actuator(s) within a time period,
g. failure of said user to respond to said alarm within said time period results in administration of said medicament

2. The method of claim 1, wherein the actuator is a momentary switch, wherein the consistent activation of said actuator is the continuous engagement of the momentary switch in a closed position, and the deactivation or inconsistent activation of said actuator is the release or failure to consistently engage said momentary switch.

3. The method of claim 1, wherein the actuator further comprises of at least two momentary switches, wherein the consistent activation of said actuator is the consistent engagement of multiple said momentary switches, and the deactivation or inconsistent activation of said actuator is the release or failure to consistently engage at least one of said momentary switches.

4. The method of claim 1, wherein the actuator further comprises of at least one switch having an open state, a closed state and an intermediate state and wherein the consistent activation of said actuator is operating said switch in said intermediate state, the deactivation of said actuator is the release or operation of said switch in said open state, and the inconsistent activation of said actuator is operating said switch in said closed state.

5. The method of claim 1, wherein the actuator is a motion detection sensor, wherein said activation of said actuator is detecting a measured amount of movement of the user within a provided threshold, and said deactivation or inconsistent activation of said actuator is movement of the user below said provided threshold.

6. The method of claim 5, wherein the motion sensor actuator is an accelerometer.

7. The method of claim 5, wherein the motion sensor actuator is a video camera.

8. The method of claim 1, wherein the actuator is a video camera using facial recognition technology and wherein the consistent activation of said actuator is detection of eyelid level, cornea or pupils above a predetermined threshold, and said deactivation or inconsistent activation of said actuator is detection of eyelid level, cornea or pupils below a predetermined threshold.

9. The method of claim 1, wherein said consistent activation of said actuator may additionally require said user to deactivate and then re-activate said actuator in response to a visual, auditory or tactile prompt.

10. The method of claim 1, wherein said consistent activation of said actuator does not require the continuous activation of said actuator but requires said user to intermittently activate said actuator in response to a visual, auditory or tactile prompt triggered on a calculated interval.

11. The method of claim 1, wherein user input for a determination of conscious or unconscious state may include a) date and time of device use, b) user response time, c) alarm frequency, d) medicament administration, and e) geographic location, which are transmitted to a central repository and analyzed to provide a feedback to said user, healthcare provider or other third parties on a) high risk drug use behaviors, b)harm reduction advice, c) consistency of device usage or d) drug tapering regimens.

12. The method of claim 11, wherein data collected from several users are aggregated and analyzed to provide a public interest data which may include local drug use data and presence of high potency or lethal batches of drugs.

13. A system for monitoring a user's level of consciousness and treating impaired consciousness comprising:
a. An actuator(s) configured for the detection of said user's level of consciousness,
b. An alarm component,
c. A battery,
d. A processing unit including a microprocessor, and a memory that interfaces with said actuators and said alarm component adapted to determining the impaired consciousness of said user,
e. A medicament administration component, containing at least one dosage of a medicament, in communication with said processing unit wherein the said component is adapted to receive a signal from the said processing unit directing the administration of said medicament on determination of said impaired consciousness of said user.

14. The system of claim 13, wherein said actuator(s) is contained within said medicament administration component housing and comprises at least one of momentary switch(es), intermediate switch(es), accelerometer or video camera.

15. The system of claim 13, wherein said actuator(s) is contained within an electronic device such as a tablet or smartphone containing sensors such as accelerometers or video cameras, and connected to the medicament housing by a wire cable or a wireless technology.

16. The system of claim 13, wherein said actuator(s) is contained in a separate housing from said medicament administration component housing and comprises at least one of momentary switch(es), intermediate switch(es), accelerometer or video camera.

17. The system of claim 13, wherein said medicament administration component comprises of a medicament autoinjector or nasal spray.

18. The system of claim 13, wherein said alarm component comprises of at least one of audio speaker(s), light emitting diode or haptic vibration component.

19. The system of claim 13, wherein said alarm component is further configured to provide a warning to the patient that medicament will be administered unless the patient reactivates actuator(s) that communicates to the system that the patient is alert.
